(19) United States
(12) Patent Application Publication
(10) Pub. No.: US 2013/0300572 A1
(43) Pub. Date: Nov. 14, 2013

(54) SYSTEM AND METHOD FOR HAND CLEANSING

(71) Applicant: Carl Nee-Kofi Mould-Millman, Atlanta, GA (US)

(72) Inventor: Carl Nee-Kofi Mould-Millman, Atlanta, GA (US)

(21) Appl. No.: 13/471,262

(22) Filed: May 14, 2012

(51) Int. Cl. G08C 19/16; G01N 33/00; G08B 23/00; B65D 83/00; G08B 21/18

(52) U.S. Cl. 340/870.01; 222/129; 340/573.1; 340/501; 73/865.8

(57) ABSTRACT

A system and method for cleansing hands is disclosed. The hand cleansing system comprises at least one sensor. The hand cleansing sensor is configured to detect whether a clinician is cleansing their hands. An identification unit is coupled with the hand cleansing sensor and is configured to determine the identity of the clinician cleansing their hands.

### BACKGROUND

About 1 in 20 hospital patients become ill with an infection. Hospital-acquired infections, also known as nosocomial infections, are the fourth leading cause of death in the United States. In the United States, the Centers for Disease Control and Prevention estimate that roughly 1.7 million hospital-acquired infections cause or contribute to 99,000 deaths each year. Many of these infections come from the hands of health care clinicians. Recently, hospitals have been given a financial incentive to address this issue. In 2008, Medicare began to stop reimbursing hospitals for the cost of treating some nosocomial infections. In some cases, hospitals may not bill patients for some hospital acquired infections. Moreover, insurance carriers may base the price of coverage in part on the prevalence of nosocomial infections.

Currently, hospitals are stymied by the task of getting health care workers to wash their hands. Most hospitals report compliance of around 40 percent. Intensive care units, where health care workers are under the most stress and patients are the sickest and most endangered by infection, usually have the lowest rates at between 30 and 40 percent.

### BRIEF DESCRIPTION OF THE DRAWINGS

The accompanying drawings, which are incorporated in and form a part of this application, illustrate embodiments of the subject matter, and together with the description of embodiments, serve to explain the principles of the embodiments of the subject matter. Unless noted, the drawings referred to in this brief description of drawings should be understood as not being drawn to scale.

FIG. 1 illustrates an example hand cleansing system, in accordance with an embodiment.

FIG. 2 illustrates an example hand cleansing system, in accordance with an embodiment.

FIG. 3 illustrates an example hand cleansing system communicatively coupled with a hospital system, in accordance with an embodiment.

FIG. 4 illustrates an example hand cleansing system, in accordance with an embodiment.

FIG. 5 illustrates an example hand cleansing system, in accordance with an embodiment.

FIG. 6 is a block diagram of an exemplary computer system used in accordance with an embodiment of the present invention.

FIG. 7 illustrates an exemplary hand cleansing system within a clinical setting, in accordance with an embodiment.

FIG. 8 illustrates an example of a plurality of hand cleansing systems within the same room, in accordance with an embodiment.

FIG. 9 illustrates an exemplary clinical setting wherein more than one room contains a hand cleansing system, in accordance with one embodiment.

FIG. 10 illustrates an exemplary clinical setting wherein hand cleansing systems are located externally from treatment rooms, in accordance with an embodiment.

FIG. 11 illustrates an exemplary setting wherein hand cleansing systems are communicatively coupled with a remote administrative station, in accordance with an embodiment.

FIG. 12 is a block diagram of an exemplary hand cleansing agent dispensing system, in accordance with an embodiment.

FIG. 13 illustrates a hand cleansing system comprising a hand cleansing agent dispensing system, in accordance with an embodiment.

FIG. 14 illustrates a hand cleansing system comprising a hand cleansing agent dispensing system, in accordance with an embodiment.

FIGS. 15A-15M illustrate a flow diagram of an example method for detecting hand cleansing, in accordance with various embodiments.

### DESCRIPTION OF EMBODIMENTS

Reference will now be made in detail to various embodiments, examples of which are illustrated in the accompanying drawings. While the subject matter will be described in conjunction with these embodiments, it will be understood that they are not intended to limit the subject matter to these embodiments. On the contrary, the subject matter described herein is intended to cover alternatives, modifications and equivalents, which may be included within the spirit and scope as defined by the appended claims. In some embodiments, all or portions of the electronic computing devices, units, and components described herein are implemented in hardware, a combination of hardware and firmware, a combination of hardware and computer-executable instructions, or the like. Such computer-executable instruction may be stored in tangible computer-readable storage media which may be fixed to or removable from the hardware of a computer system. Some examples of such tangible computer-readable storage media include, but are not limited to, silicon based memory, solid state or disk-based storage drives, and/or an optical storage disk. Furthermore, in the following description, numerous specific details are set forth in order to provide a thorough understanding of the subject matter. However, some embodiments may be practiced without these specific details. In other instances, well-known methods, procedures, objects, and circuits have not been described in detail as not to unnecessarily obscure aspects of the subject matter.

### Notation and Nomenclature

Unless specifically stated otherwise as apparent from the following discussions, it is appreciated that throughout the present Description of Embodiments, discussions utilizing terms such as “determining,” “alarming,” “outputting,” “sending,” “timing,” “dispensing,” “detecting,” “placing,” “prohibiting,” “disarming,” “reporting,” or the like, refer to the actions and processes of a computer system or similar electronic computing device (or portion thereof) such as, but not limited to: a processor, a display device, and/or an I/O device. The electronic computing device manipulates and transforms data represented as physical (electronic) quantities within the electronic computing device's processors, registers, and/or memories into other data similarly represented as physical quantities within the electronic computing device's memories, registers and/or other such information storage, processing, transmission, or/or display components of the electronic computing device or other electronic computing device(s).

### Overview of Discussion

Example techniques, devices, systems, and methods for hand cleansing are described herein. High level discussion of an example hand cleansing system is then described. Discussion continues with a description of example hand washing protocols and methods of determining whether hand washing protocols are followed by clinicians. Next, devices in the hand cleansing system are described including cleansing agent dispensers, identification systems, input/output devices, telematics devices, alarm units, and an example computer system environment. Discussion continues with an example of how hand cleansing systems can be integrated in clinical settings. Next, the way in which hand cleansing data is analyzed and distributed is discussed. Lastly, example methods of use are discussed.

### Example Hand Cleansing System

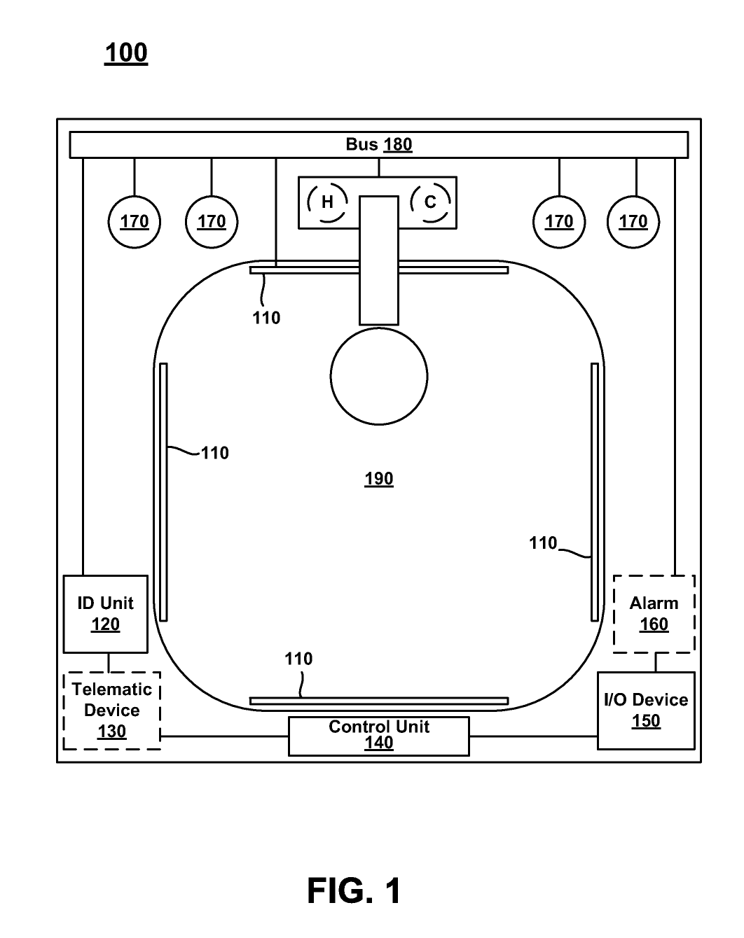

FIG. 1 is an example of hand cleansing system 100, according to one embodiment. In one embodiment, hand cleansing system 100 comprises a sink 190 with various components embedded therein. In some embodiments, the components may include hand cleansing sensors 110, hand cleansing agent dispensers 170, an identification unit 120, a telematics device 130, a control unit 140, an input/output (“I/O”) device 150, and a communications bus 180. In some embodiments, water may run through hand cleansing system 100, regardless of whether sink 190 is a legacy sink or otherwise.

In order to increase compliance with hand cleansing protocols by providing accountability, in some embodiments clinicians 700 are detected and identified by identification unit 120. Identification unit 120 identifies a clinician, and in some embodiments records the identification in a system memory. According to some embodiments, identification unit 120 can also detect whether clinician 700 is in a room with hand cleansing system 100. Additionally, in some embodiments, identification unit 120 can determine the distance between hand cleansing system 100 and clinician 700.

A clinician 700 is not limited to a physician. For example, a clinician could be a registered nurse, a licensed practical nurse, resident, medical student, staff member, or anyone needed in or around a care facility. In some embodiments, clinician 700 could be a visitor such as a family member or administrator.

In some embodiments hand cleansing system 100 comprises at least one alarm 160. Alarm 160 may be configured to alert a clinician 700 when they have not cleansed their hands In some embodiments, Alarm 160 is coupled with identification unit 120 and control unit 140. Alarm 160 may be triggered when clinician 700 enters a room and does not cleanse their hands. In some embodiments, Alarm 160 may be triggered when clinician 700 uses the wrong cleansing agent, fails to cleanse their hands for the required amount of time, or otherwise fails to follow a hand cleansing protocol. In some embodiments, alarm 160 is located on or within hand cleansing system 100. In other embodiments alarm 160 is located remotely from hand cleansing system 100. In some embodiments, a clinician may disarm alarm 160. In other embodiments someone other than clinician 700 is required to disarm alarm 160.

In some embodiments hand cleansing system 100 comprises at least one I/O device 150 as further described herein. I/O device 150 can take many forms. For input purposes, I/O device 150 may comprise a keypad, touchpad, microphone, pad for use with a stylus pen, QWERTY keyboard, mouse, trackball, etc. For output, I/O device 150 may comprise a graphical user interface, a liquid crystal display, speaker, etc. In some embodiments, I/O device 150 comprises an interface configured to couple with data storage devices such as a USB flash drive, floppy disk, CD-ROM, etc. Data may be imported or exported through I/O device 150. In addition, in some embodiments, I/O device 150 may be used to program, or change the configuration of hand cleansing system 100.

In some embodiments, control unit 140 is configured to coordinate the operation of some or all of: sink 190, identification unit 120, sensors 110, alarm 160, I/O device 150, hand cleansing agent dispensers 170, and telematic device 130. In some embodiments control unit 140 may comprise a computer system 600. In some embodiments, hand cleansing system 100 may comprise a bus 180 coupled with the various components of hand cleansing system 100. In some embodiments control unit 140 may control the temperature of water dispensed from sink 190.

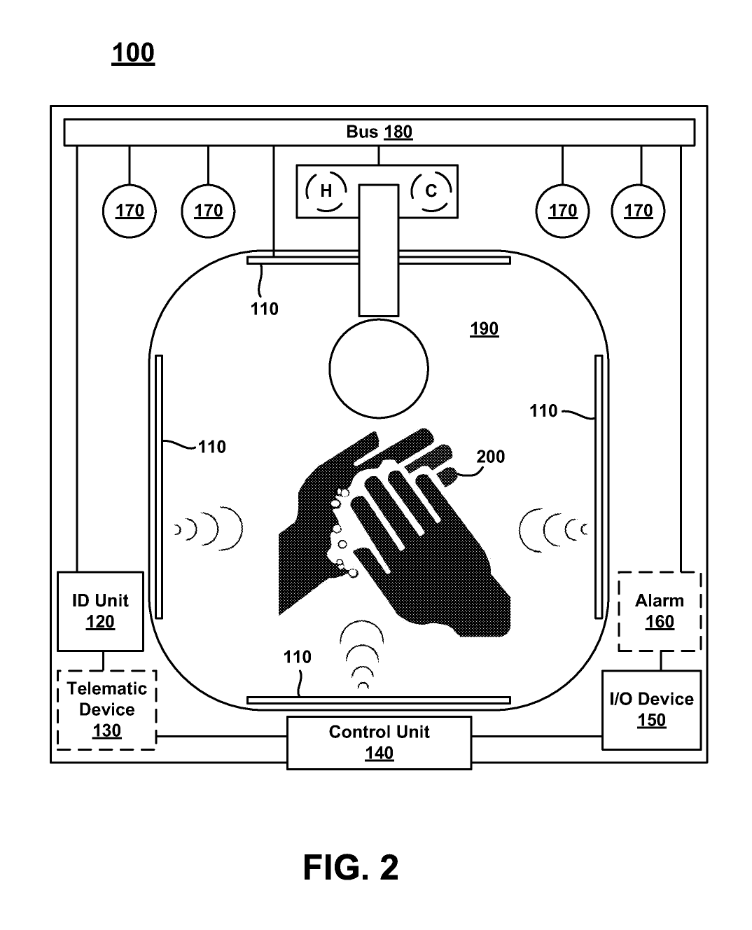

With reference to FIG. 2, in some embodiments hand cleansing sensors 110 detect motion, configured to ensure clinician 700 is washing one or both of their hands. In some embodiments sensors 110 may be optical sensors, rather than motion detectors. In other embodiments, as described herein, sensors 110 can detect volatile compounds on clinician 700 hands. In some embodiments, sensors 110 can detect whether clinician 700 is using the proper technique when cleansing their hands.

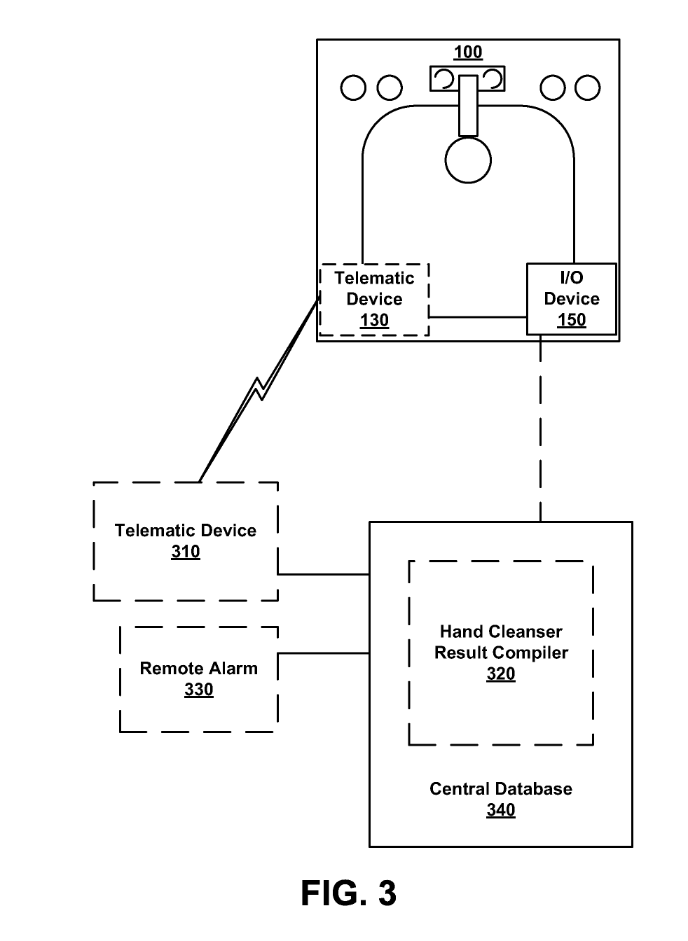

With reference to FIG. 3, in some embodiments hand cleansing system 100 comprises at least one telematic device 130. In some embodiments telematic device 130 is communicatively coupled with a central database 340 as described further herein. Telematic device 130 can be configured to send and receive data to and from a central database. In other embodiments, instead of telematic device 130, I/O device 150 may be coupled, communicatively or otherwise with central database 340.

Still referring to FIG. 3, in some embodiments telematic device 130 may be communicatively coupled with another telematic device 310 which in turn is coupled with central database 340. As a few examples, central database 340 may be located at a nurse's station, an administrative office, or remotely from the building where hand cleansing system 100 is located. Central database 340, as further described herein, may comprise a hand cleansing data compiler 320. In some embodiments alarm 160 may be coupled with central database 340.

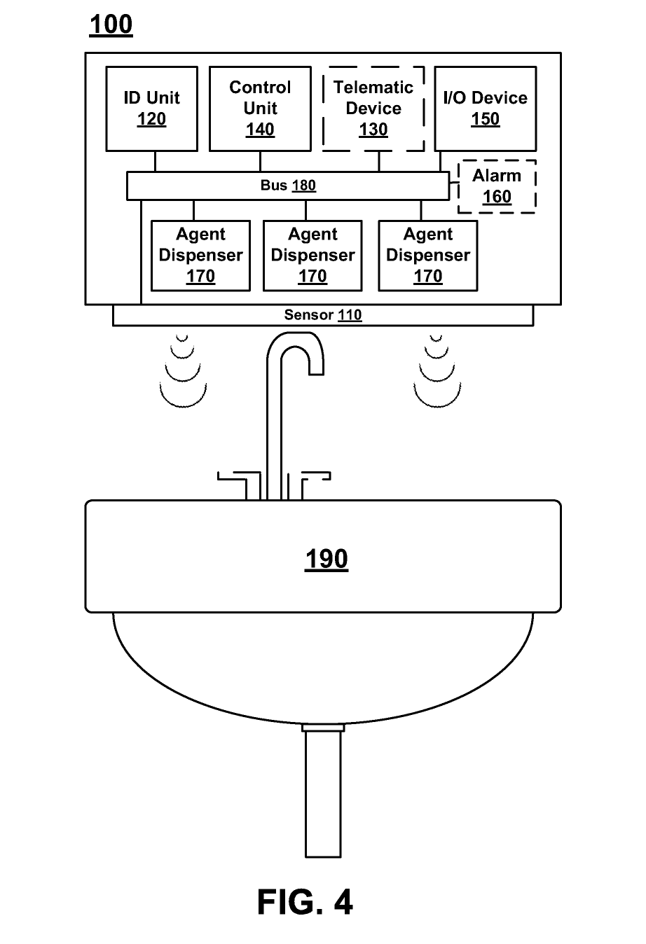

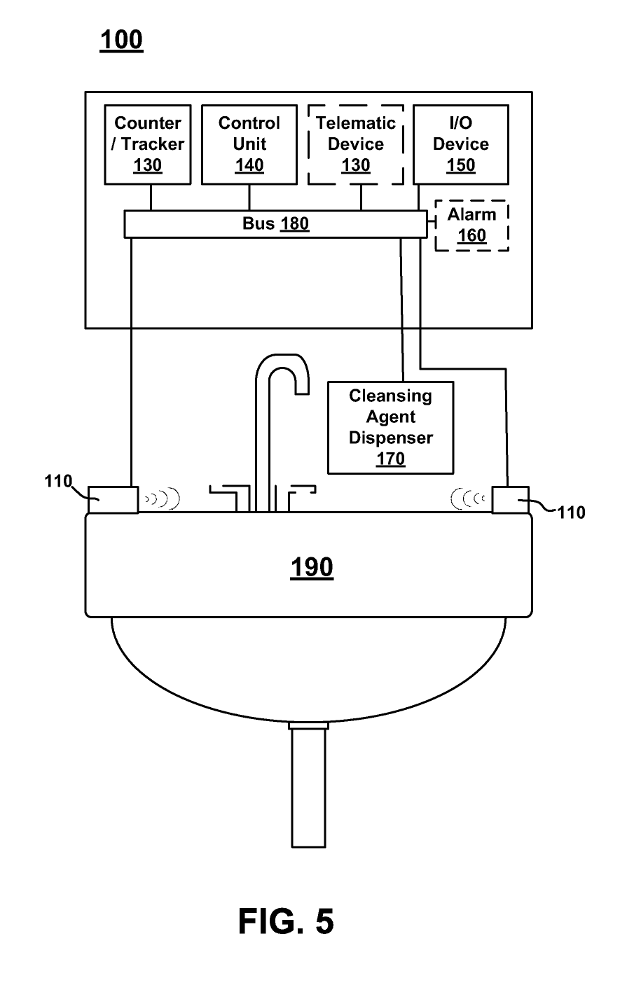

In some embodiments, some or all components are built into sink 190. As illustrated in FIGS. 4 and 5, in some embodiments, sink 190 is a legacy sink and not specifically built to support hand cleansing system 100. In some embodiments, hand cleansing system 100 may be a wall-mounted unit configured for use with a legacy sink 190. In other embodiments it hand cleansing system may snap on the side of legacy sink 190. FIG. 4 illustrates hand cleansing system 100 configured for use with a legacy sink 190.

### Example Hand Cleansing Protocols

Hand cleansing system 100 is intended to combat the spread of infection. As such, in some embodiments, identification unit 120 can detect where clinician 700 has recently been. For example, hand cleansing system 100 can detect whether a clinician recently used a restroom, recently entered a health facility, or recently entered a particular room. In some embodiments, identification unit can detect whether a clinician has recently visited a particular patient or hospital bed 730. In some embodiments central database 340 tracks the movement of clinician 700. In some embodiments identification units 120 are positioned throughout a facility such that central database 340 can record and analyze the movements and patterns of clinician 700. In some embodiments, hand washing system 100 may base how a clinician must cleanse their hands at least in part on where clinician 700 has been.

In some embodiments, sensors 110 can detect if clinician 700 does not follow a hand cleansing protocol. In some embodiments hand cleansing system 100 may comprise a timer, to determine the amount of time a clinician 700 spends cleansing their hands. In some embodiments, sensors 110 can detect whether clinician 700 did not wash their hands for certain period of time, washed their hands in a wrong direction, did not use the correct soap, etc. In some embodiments sensors 110, alone or in conjunction with other components of hand cleansing system 100, can detect whether the hands of a clinician 700 have been wet with running water, whether soap has been applied, the amount of time spent lathering, the amount of time spent rinsing, etc. In some embodiments, sensors 110 compare how a clinician 700 washes their hands with heuristic models. In some embodiments sensors may be configured to determine whether the clinician 700 washed: each finger, in between each finger, the bottom of hands, the top of hands, wrists, under fingernails, etc.

### Cleansing Agent Dispensers

Nosocomial infections due to resistant organisms are a serious problem that threatens patients. Microbes can acquire resistance to antibiotics, antifungal, and antiviral agents. As the numbers of resistant organisms increases, the number of new antimicrobial agents required to treat them has not kept pace. In some embodiments, if the wrong cleansing agent 1220 is dispensed alarm 160 may be triggered.

In some embodiments, cleansing agent dispenser 170 dispenses cleansing agents intended to reduce infection. Cleansing agent dispenser 170 may comprise a pump and container containing cleansing agent 1220.

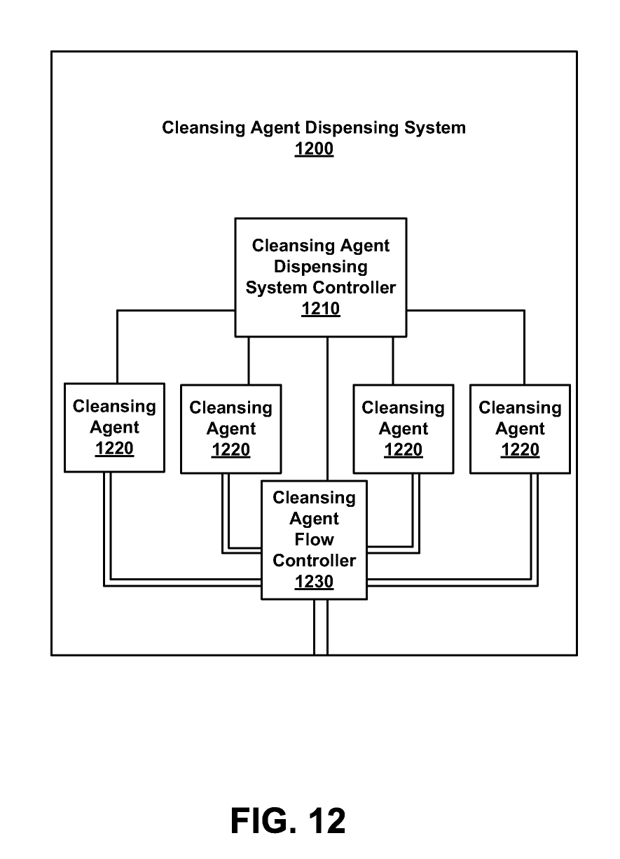

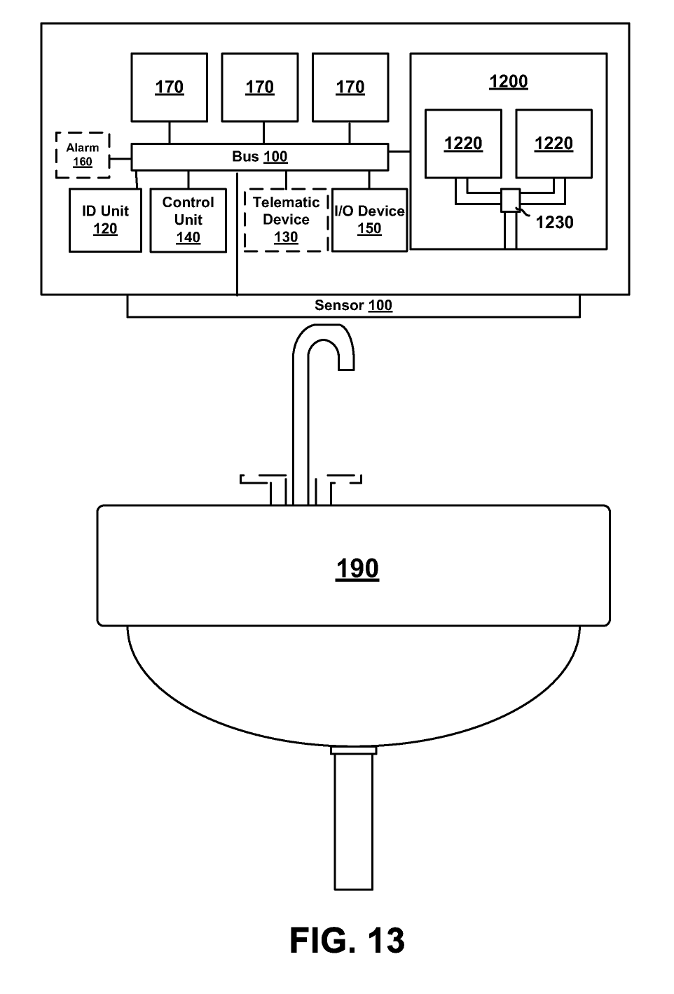

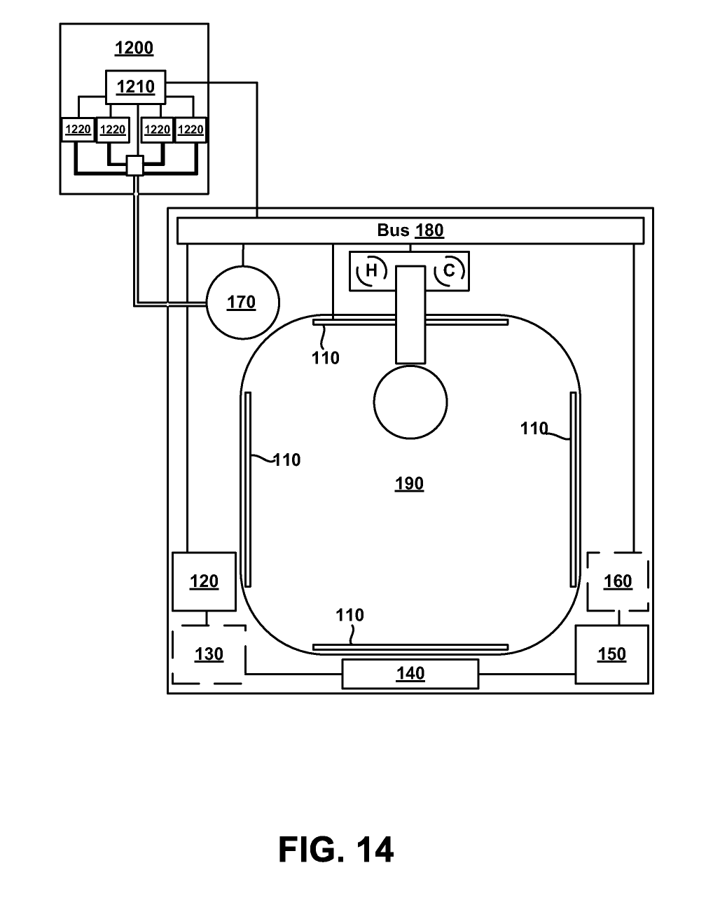

FIG. 12 illustrates an example cleansing agent dispensing system comprising a cleansing agent dispensing system controller 1210, a cleansing agent flow controller 1230, and a plurality of cleansing agents 1220. In some embodiments cleansing agent dispenser 170 may be coupled with a cleansing agent dispensing system 1200. As illustrated in FIG. 13, in some embodiments hand cleansing system 100 comprise a plurality of hand cleansing dispensers 170 and cleansing agent dispensing systems 1200. With reference to FIG. 14, in some embodiments a cleansing agent dispensing system may be fluidly coupled with a cleansing agent dispenser 170.

In some embodiments, cleansing agent dispensing controller 1210 is configured to dispense a particular cleansing agent 1220, or a combination of cleansing agents 1220. Cleansing agent dispensing system controller 1210 is further configured to control the cleansing agent flow controller 1230. Cleansing agent flow controller 1230, in some embodiments, is configured to dispense the correct amount of cleansing agent 1220. In some embodiments, cleansing agent flow controller 1210 is configured to prevent the incorrect cleansing agent 1220 from being dispensed.

In some embodiments, cleansing agent 1220 may comprise various types of soap. In some embodiments, soap includes detergent based products that contain esterified fatty acids and sodium or potassium hydroxide. In some embodiments, hand cleansing system 100 comprises liquid soaps. In other embodiments soaps may take the form of bar soap, tissues, or leaflets. In some embodiments, soaps are intended to remove dirt, soil, and various organic compounds from the hands of clinician 700.

In some embodiments, cleansing agents 1220 may include alcohols. Alcohol-based antiseptics may contain isopropanol, ethanol, n-propanol, or a combination thereof. Other solutions may contain, alone or in combination: hexachlorophene, quaternary ammonium compounds, povidone-iodine, triclosan, octenidine dihydrochloride, chlorhexidine gluconate (“CHG”), etc. Chlorhexidine gluconate attaches to, and subsequently disrupts the cytoplasmic membranes of microbes resulting in the precipitation of their cellular contents. Chlorohexylenol can also be used as a cleansing agent 1220 as it inactivates bacterial enzymes and alters their cell walls. In addition ethylene-diaminetetraacetic acid may be used in cleansing agent solutions 1220. It should be understood that other alcohol-based cleansing agents 1220 may be used in hand cleansing system 100. In some embodiments, cleansing agents 1220 may include antiseptics that prevent the formation of biofilms when dispensed. In some embodiments, cleansing agents 1220 may include antiseptics that are anti-microbial and/or anti-irritative.

### Identification Systems

Hand cleansing system 100 is configured to identify clinicians through identification unit 120. Clinician 700 identification can be implemented using a variety of methods. For example, identification unit 120 may comprise a radio frequency identification (“RFID”) reader to identify and track clinician 700. In such a case, some type of taggant such as a microchip may be embedded in the badge of a clinician 700.

In some embodiments other types of identification may be used. In some embodiments a clinician 700 may be required to enter a unique password using the I/O device 150. For example, each clinician 700 may have a password they must enter before using hand cleansing system 100. In some embodiments, they a keyboard or touch screen may used to enter a password. In one embodiment, a touch screen with a stylus may be configured such that a clinician 700 must sign their name to verify their identity.

In some embodiments, biometrics may be used to identify a clinician 700. For example, hand cleansing system 100 may comprise an I/O device which reads fingerprints. In one embodiment, I/O device may identify a clinician 700 by their voice, or by scanning their iris.

### Input/Output Devices

In some embodiments hand cleansing system 100 comprises at least one I/O device. I/O devices can perform a number of functions, including but not limited to: identifying clinicians 700, programming various functions of hand cleansing system 100, programming hand cleansing dispensers 170 or hand cleanser dispensing system 1200, transmitting data corresponding with hand cleansing to central database 340, programming water temperature, providing data to clinicians through a disk drive or the like, disarming alarm 160, and programming alarm 160.

In some embodiments, input devices may include, but are not limited to: keypads, keyboards, virtual keyboards, touchscreens, touchscreens configured for receiving input from a stylus, pointing devices, mice, joysticks, composite input devices, cameras, scanners, laser rangefinders, image scanners, disk drives, telematic devices, receivers, and microphones.

In some embodiments, I/O device 150 may be configured for programming hand cleansing device 100. For example, a keyboard may give an operator the ability to program particular configurations and settings for hand cleansing system 100. These configurations and settings may include, but are not limited to: the temperature of the water, the amount of cleansing agent to be dispensed, various hand washing protocols, when to trigger an alarm, where to trigger an alarm, when to transmit data to central database 340, etc. It should be understood that in some embodiments I/O device 150 may comprise a disk drive, telematic device, or other receiver, which transfers data regarding configurations and settings to hand cleansing system 100.

Further, in some embodiments an I/O device 150 may be used alone or in conjunction with identification unit 120 to identify a clinician 700. In some embodiments, a clinician 700 may be required to enter a unique password before using hand cleansing system 100. In some embodiments, clinician 700 or someone else may be required to enter a password on I/O device 150 to deactivate alarm 160.

In some embodiments, output devices include, but are not limited to: monitors, speakers, alarms, and touchscreens. In some embodiments a graphical user interface (“GUI”) communicate to clinician 700 how they should cleanse their hands. For example, I/O device 150 may tell clinician 700 that they must wash their hands for a particular amount of time while using a particular cleansing agent. In some embodiments, clinician 700 must communicate that they understand with I/O device 150.

### Telematic Devices

In some embodiments, hand cleansing system 100 may include a telematic device 130. Telematic devices 130 may transmit or receive information wirelessly. In some embodiments, telematic device 130 is communicatively coupled with another telematic device 310, or directly to central database 340. In some embodiments telematic device 130 may receive information regarding configuration of hand cleansing system 100. In some embodiments telematic device 130 transmits a signal triggering remote alarm 160. In some embodiments telematic device 130 transmits data corresponding to hand cleansing to hand cleanser result compiler 320. It is to be understood that telematic device 130 can be used perform other functions described herein.

### Example Alarm Units

In some embodiments, hand cleansing system 100 comprises an alarm 160. For the purposes of this discussion, disarming and deactivating alarm 160 are equivalents. In some embodiments alarm 160 is a device or system configured to give an audible or visual alarm signal. Alarm 160 may take various forms. For example, alarm 160 may comprise a light, a bell, a tickler, etc. In some embodiments, alarm 160 may be integrated in hand cleansing system 100. In some embodiments, alarm 160 may be installed in sink 190. In some embodiments, alarm 160 may be configured to send a message to a personal device such as a pager, cellular phone, or other wireless device belonging to clinician 700.

In some embodiments alarm 160 is located remotely from hand cleansing system 100. For example, an alarm may be located near a nurse's station or other administration location. In such an example, after triggering alarm 160, clinician 700 or someone near alarm 160 must disarm alarm 160. In some embodiments, alarm 160 requires someone other than clinician 700 to disarm alarm 160. In some embodiments, someone other than clinician 700 may be required to identify themselves and observe clinician 700 following the correct hand cleansing protocol. For example, after a clinician 700 triggers an alarm, a nurse may need to go to hand cleansing system 100, identify herself to hand cleansing system 100, clinician 700 must cleanse their hands again, and the nurse may need to sign off to tell hand cleansing system 100 that clinician 700 did in fact cleanse their hands.

A variety of events may trigger alarm 160. In some embodiments, alarm 160 may be triggered when a clinician 700 fails to follow a particular hand cleansing protocol. Other events triggering alarm 160 include, but are not limited to: entering a room and not using hand cleansing system 100 within a particular amount of time, clinician 700 failing to properly cleanse their hands 200, clinician 700 failing to wash their hands 200 for a particular duration, clinician 700 moving away from hand cleansing system 700 while hand cleansing system 700 is in use, etc.

### Example Computer System Environment

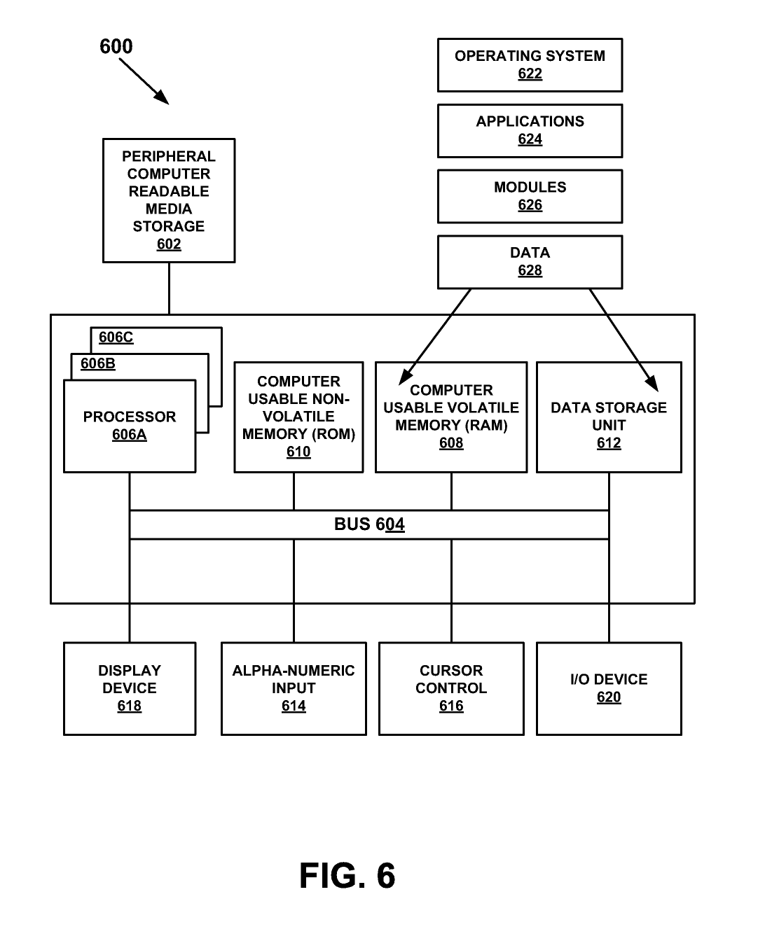

With reference now to FIG. 6, all or portions of some embodiments described herein are composed of computer-readable and computer-executable instructions that reside, for example, in computer-usable/computer-readable storage media of a computer system. That is, FIG. 6 illustrates one example of a type of computer (computer system 600) that can be used in accordance with or to implement various embodiments which are discussed herein. It is appreciated that computer system 600 of FIG. 6 is only an example and that embodiments as described herein can operate on or within a number of different computer systems including, but not limited to, general purpose networked computer systems, embedded computer systems, routers, switches, server devices, client devices, various intermediate devices/nodes, stand alone computer systems, media centers, handheld computer systems, multi-media devices, and the like. Computer system 600 of FIG. 6 is well adapted to having peripheral tangible computer-readable storage media 602 such as, for example, a floppy disk, a compact disc, digital versatile disc, other disc based storage, universal serial bus “thumb” drive, removable memory card, and the like coupled thereto. The tangible computer-readable storage media is non-transitory in nature.

System 600 of FIG. 6 includes an address/data bus 604 for communicating information, and a processor 606A coupled with bus 604 for processing information and instructions. As depicted in FIG. 6, system 600 is also well suited to a multi-processor environment in which a plurality of processors 606A, 606B, and 606B are present. Conversely, system 600 is also well suited to having a single processor such as, for example, processor 606A. Processors 606A, 606B, and 606C may be any of various types of microprocessors. System 600 also includes data storage features such as a computer usable volatile memory 608, e.g., random access memory (RAM), coupled with bus 604 for storing information and instructions for processors 606A, 606B, and 606C. System 600 also includes computer usable non-volatile memory 610, e.g., read only memory (ROM), coupled with bus 604 for storing static information and instructions for processors 606A, 606B, and 606C. Also present in system 600 is a data storage unit 612 (e.g., a magnetic or optical disk and disk drive) coupled with bus 604 for storing information and instructions. System 600 also includes an optional alphanumeric input device 614 including alphanumeric and function keys coupled with bus 604 for communicating information and command selections to processor 606A or processors 606A, 606B, and 606C. System 600 also includes an optional cursor control device 616 coupled with bus 604 for communicating user input information and command selections to processor 606A or processors 606A, 606B, and 606C. In one embodiment, system 600 also includes an optional display device 618 coupled with bus 604 for displaying information.

Referring still to FIG. 6, optional display device 618 of FIG. 6 may be a liquid crystal device, cathode ray tube, plasma display device or other display device suitable for creating graphic images and alphanumeric characters recognizable to a user. Optional cursor control device 616 allows the computer user to dynamically signal the movement of a visible symbol (cursor) on a display screen of display device 618 and indicate user selections of selectable items displayed on display device 618. Many implementations of cursor control service 616 are known in the art including a trackball, mouse, touch pad, joystick or special keys on alphanumeric input device 614 capable of signaling movement of a given direction or manner of displacement. Alternatively, it will be appreciated that a cursor can be directed and/or activated via input from alphanumeric input device 614 using special keys and key sequence commands. System 600 is also well suited to having a cursor directed by other means such as, for example, voice commands. System 600 also includes an I/O device 620 for coupling system 600 with external entities. For example, in one embodiment, I/O device 620 is a modem for enabling wired or wireless communications between system 600 and an external network such as, but not limited to, the Internet.

Referring still to FIG. 6, various other components are depicted for system 600. Specifically, when present, an operating system 622, applications 624, modules 626, and data 628 are shown as typically residing in one or some combination of computer usable volatile memory 608 (e.g., RAM), computer usable non-volatile memory 610 (e.g., ROM), and data storage unit 612. In some embodiments, all or portions of various embodiments described herein are stored, for example, as an application 624 and/or module 626 in memory locations within RAM 608, computer-readable storage media within data storage unit 612, peripheral computer-readable storage media 602, and/or other tangible computer-readable storage media.

### Example Hand Cleansing System Integration in a Clinical Setting

Described herein are examples of hand cleansing system 100 configured for clinical use. It should be understood that hand cleansing system 100 may be used elsewhere including, but not limited to: laboratories, universities, office buildings, blood clinics, dialysis centers, tattoo parlors, and homes.

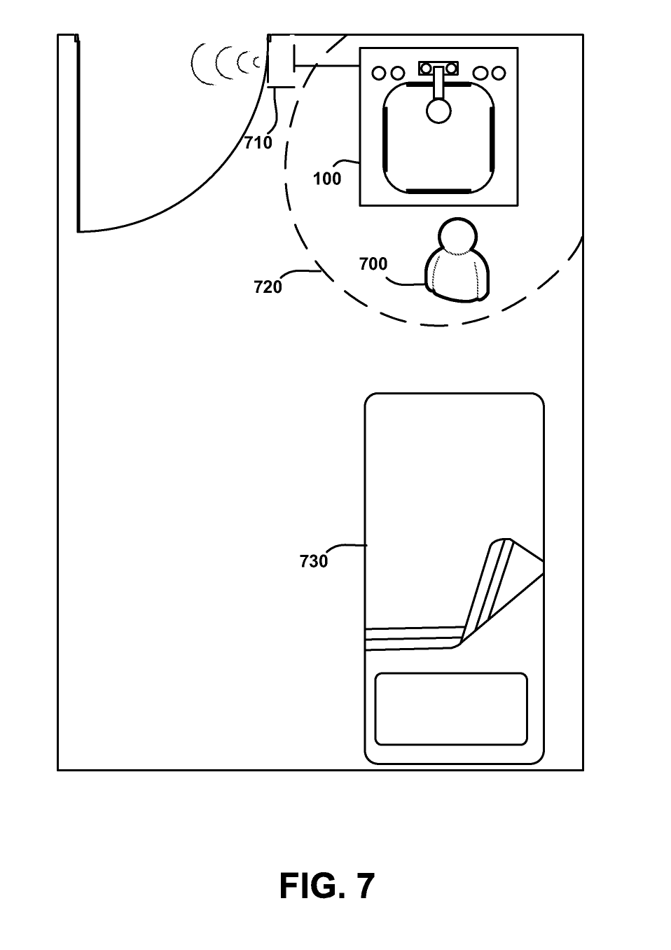

FIG. 7 illustrates a hand cleansing system in a clinical setting. In some embodiments, hand cleansing system 100 may be coupled with a room sensor 710 which determines whether someone entered a particular room. In some embodiments, when room sensor 710 determines someone entered a room, identification unit 120 determines whether the person is a clinician 700, and whether clinician 700 needs to cleanse their hands 200. As illustrated in FIG. 7, in some embodiments hand cleansing system 100 uses identification unit 120 or I/O device 150 to determine whether a physician is within a particular distance 720 from hand cleansing system 100. In some embodiments, hand cleansing system 100 is configured to determine the distance between hand cleansing system 100 and clinician 700.

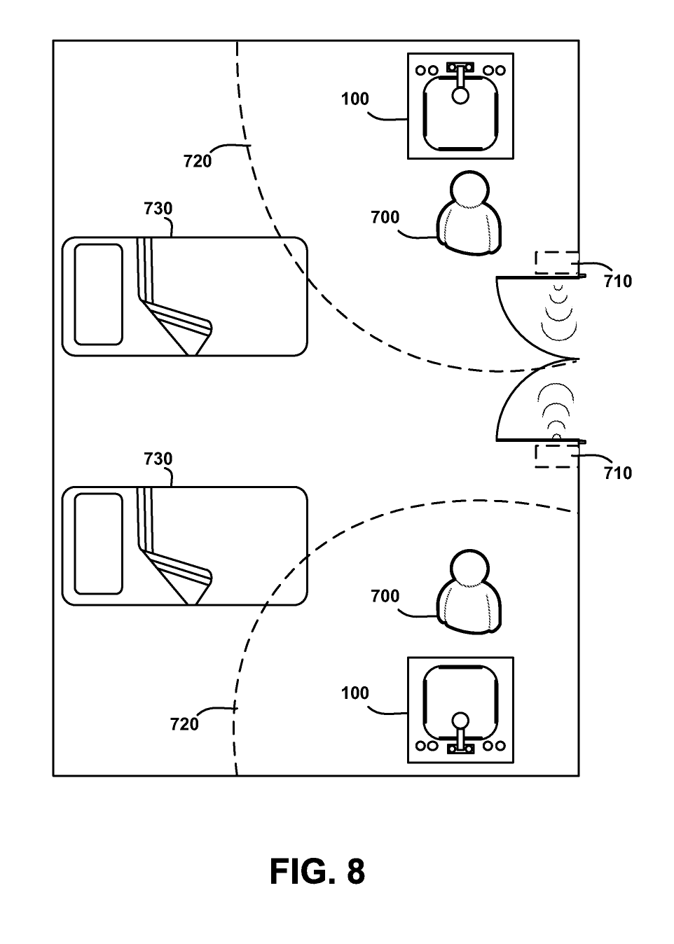

In some embodiments, a plurality of hand cleansing systems 100 may be communicatively coupled such that all hand cleansing performed in a clinic may be monitored. As illustrated in FIG. 8, in some embodiments, a room may contain more than one hand cleansing system 100. In such a case, hand cleansing systems 100 are configured such that identification units 120 and distance determinators on a first hand cleansing system 100 do not interfere with identification units, distance determinators, and other components on a second hand cleansing system.

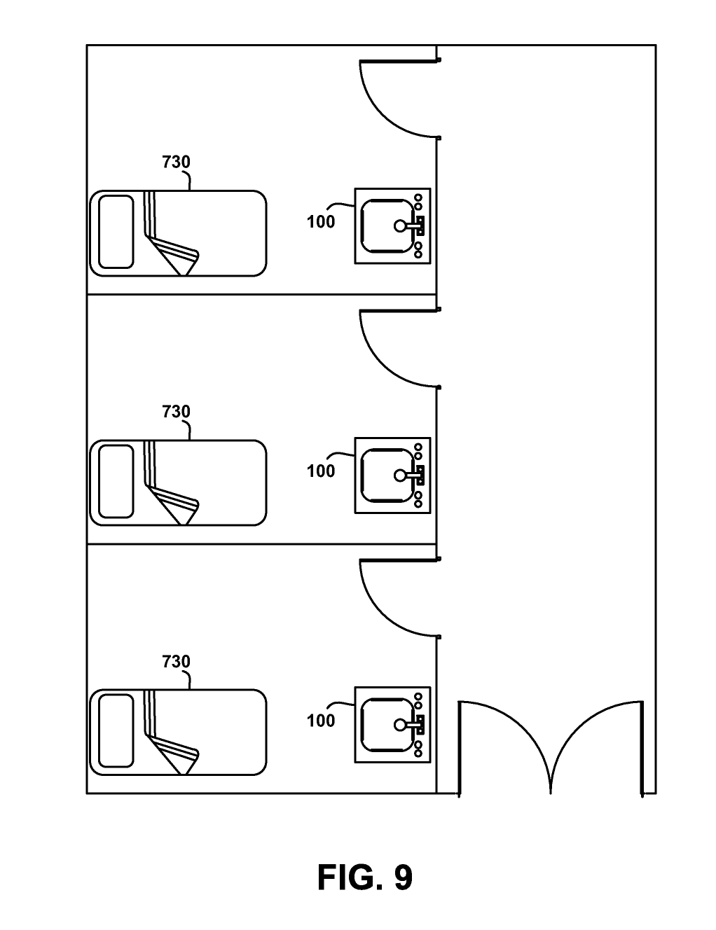

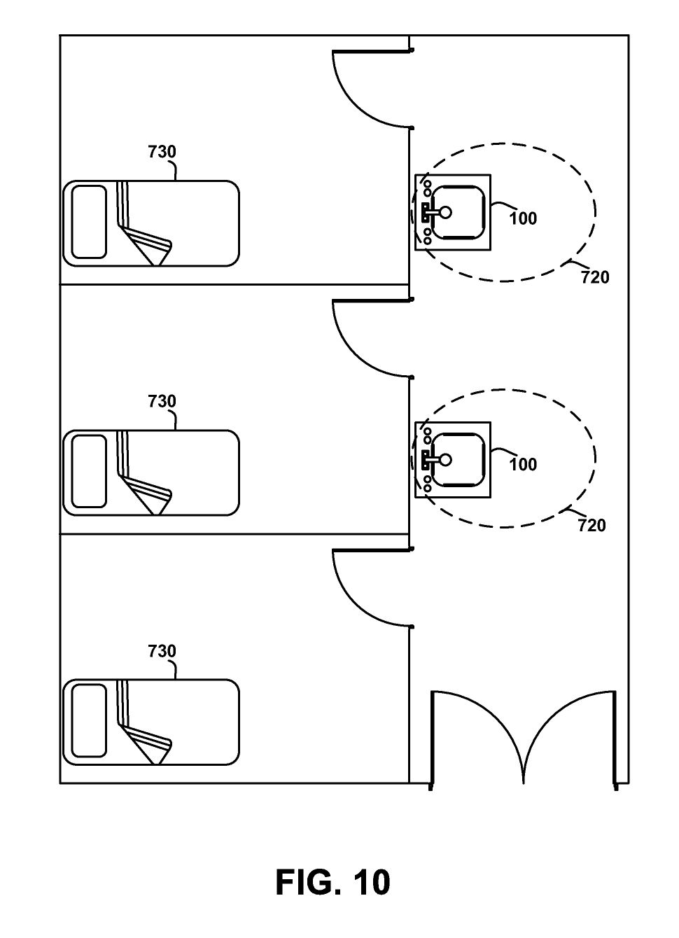

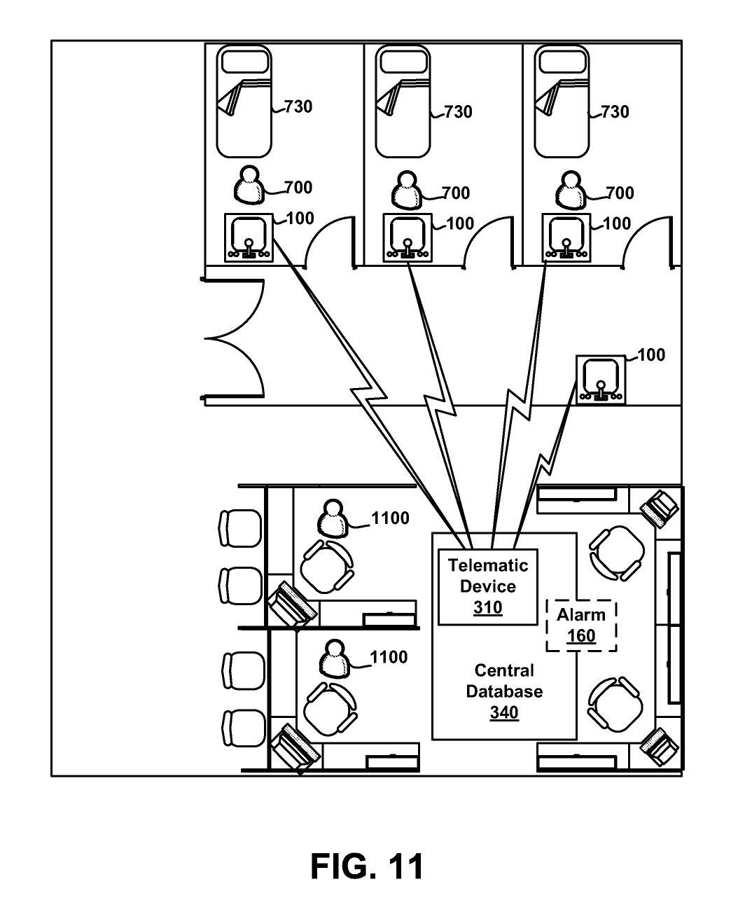

As illustrated in FIG. 9, in some embodiments, each room in a clinic may contain at least one hand cleansing system 100. With reference to FIG. 10, in other embodiments, hand cleansing systems 100 may be located outside of rooms. This can both save money and space. It is to be understood that hand cleansing systems may be placed both outside and inside of rooms as illustrated in FIG. 11.

FIG. 11 illustrates an embodiment where hand cleansing systems 100 transmit data to a central database 340. With reference to FIG. 11 and discussed herein, in some embodiments central database 340 and alarm 160 may be located at a nurse's station.

### Hand Cleansing Data Analysis

As discussed herein, in some embodiments central database 340 alone, or in conjunction with hand cleanser result compiler 320, compiles and analyzes data received from hand cleansing systems 100. In some embodiments, clinicians, administrators, and insurance carriers can access central database 340 to determine the quality of hand cleansing occurring within a particular facility or plurality of facilities. In some embodiments central database 340 or hand cleansing system 100 prints a report for each clinician correlating with their hand cleansing.

In some embodiments, central database 340 gathers information from hand washing systems 100 including, but not limited to: which clinicians 700 were in a facility, which clinicians 700 used which hand cleansing systems 100, which clinicians 700 triggered an alarm 160, how long a particular clinician 700 spent washing their hands 200, how long on average a particular clinician 700 spends washing their hands 200, how long on average all clinicians 700 spend washing their hands, what cleansing agent 1220 was used at a particular hand cleansing system 100 at a particular time, how much cleansing agents 1220 are used on average, the amount of cleansing agents 1220 in a particular hand washing system, etc.

### Example Methods of Use

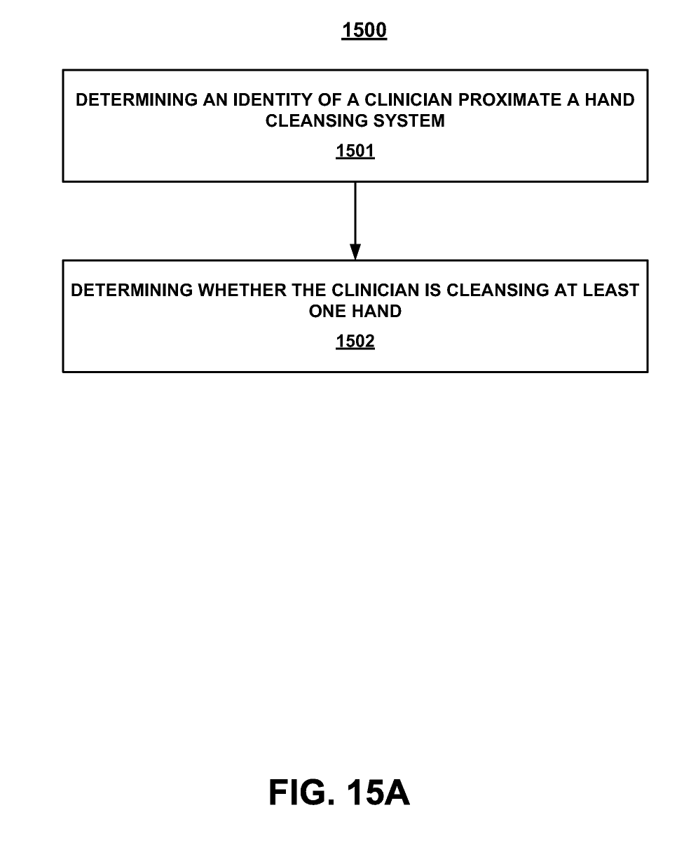

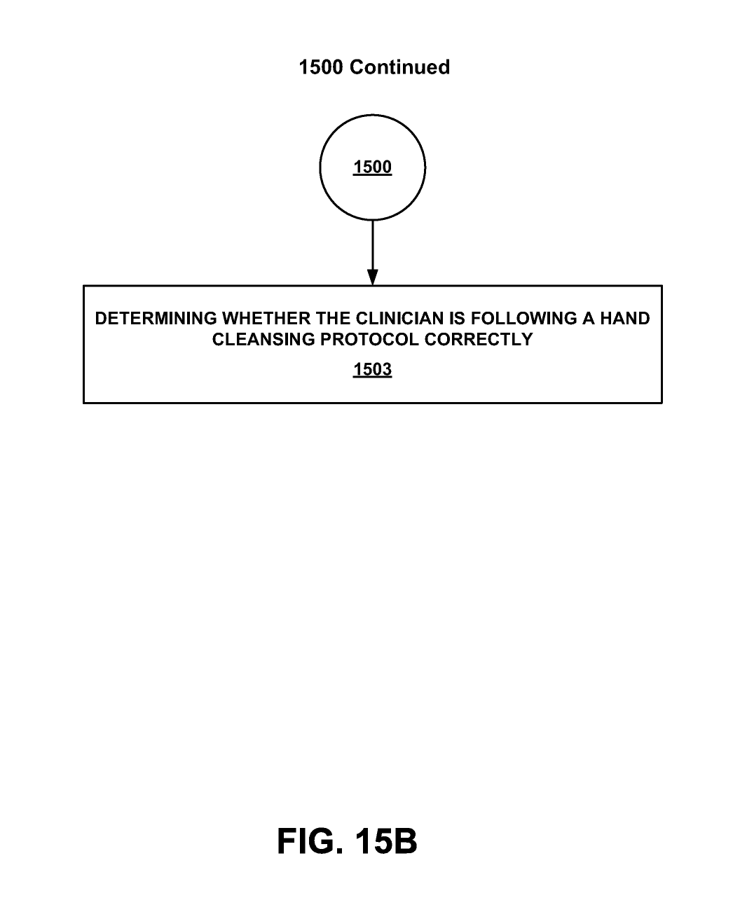

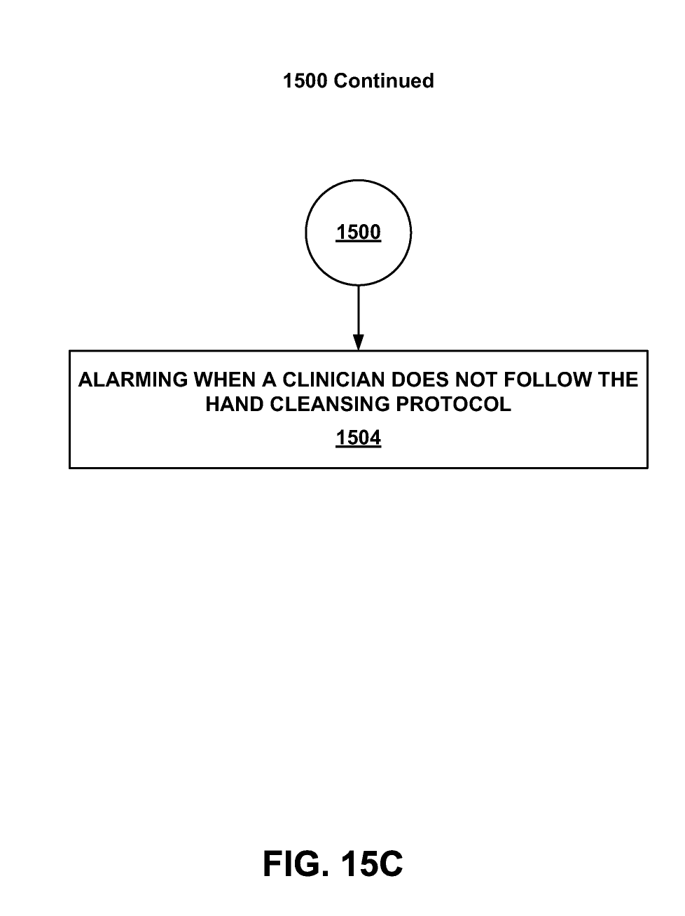

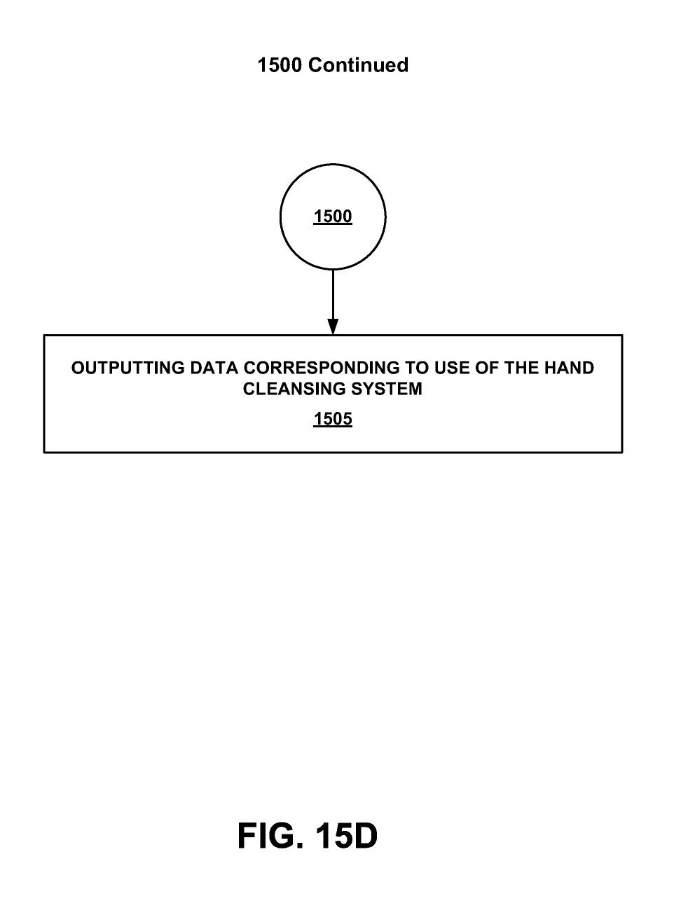

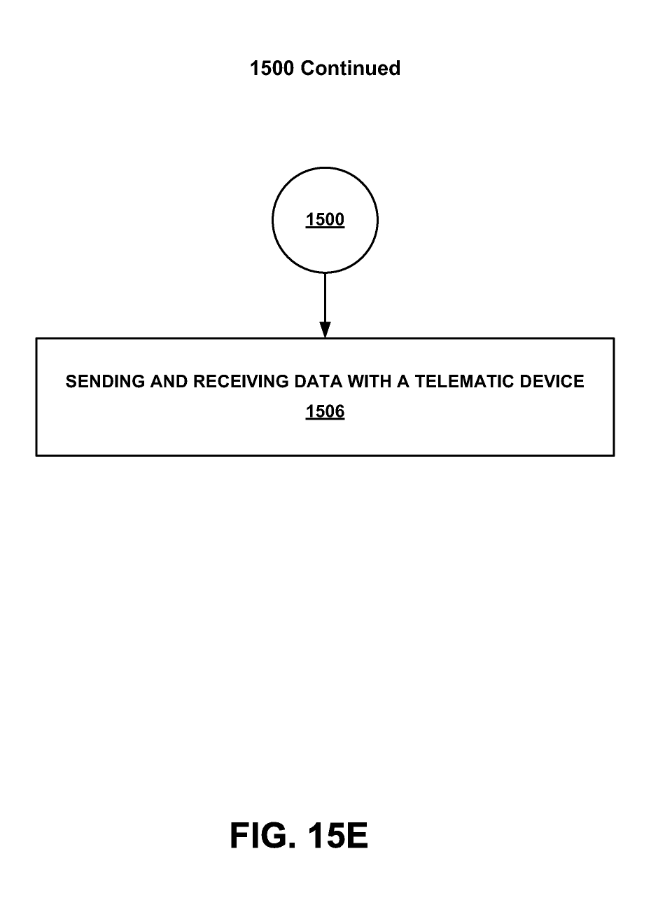

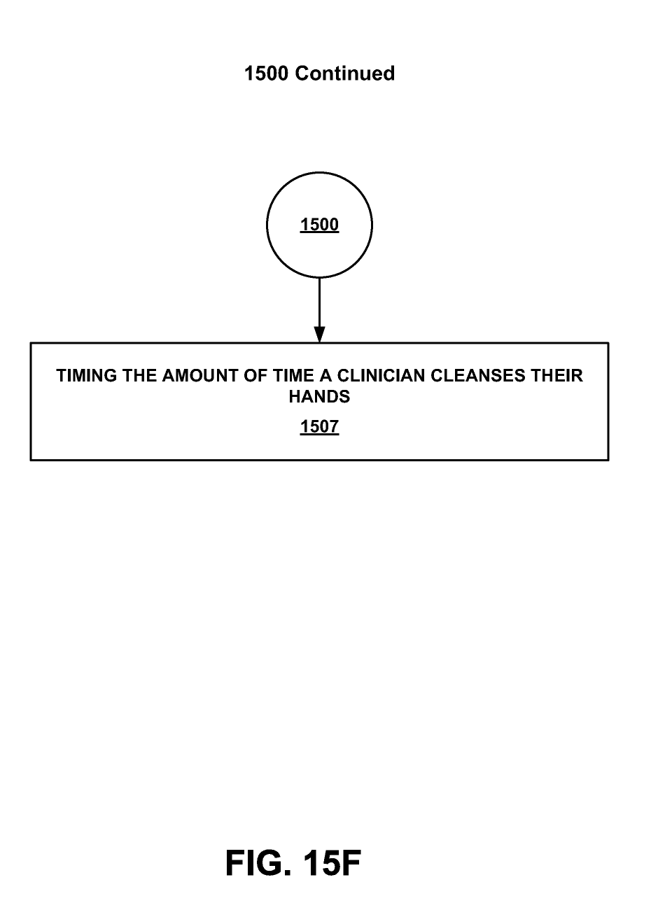

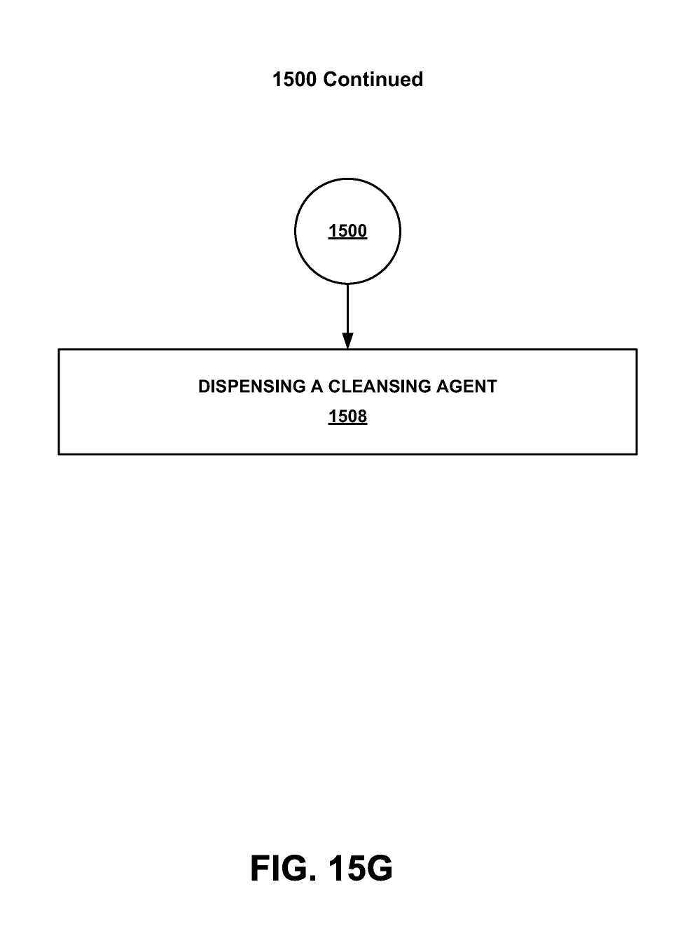

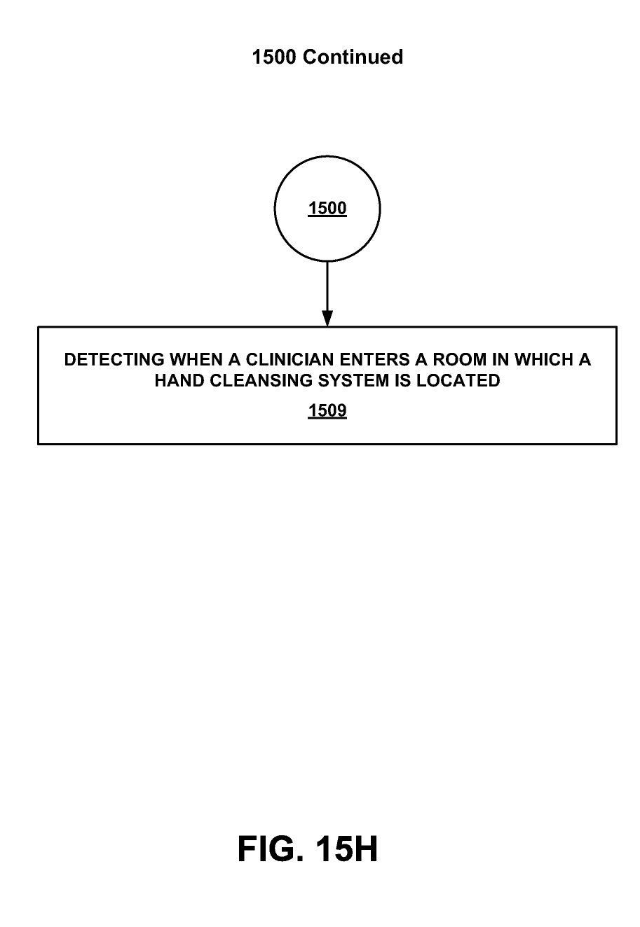

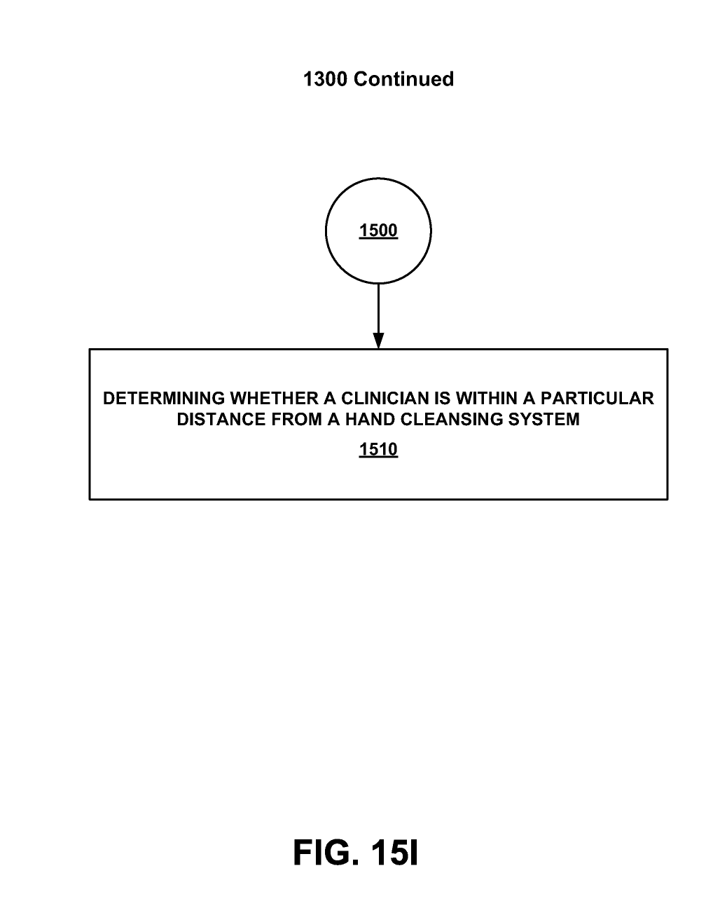

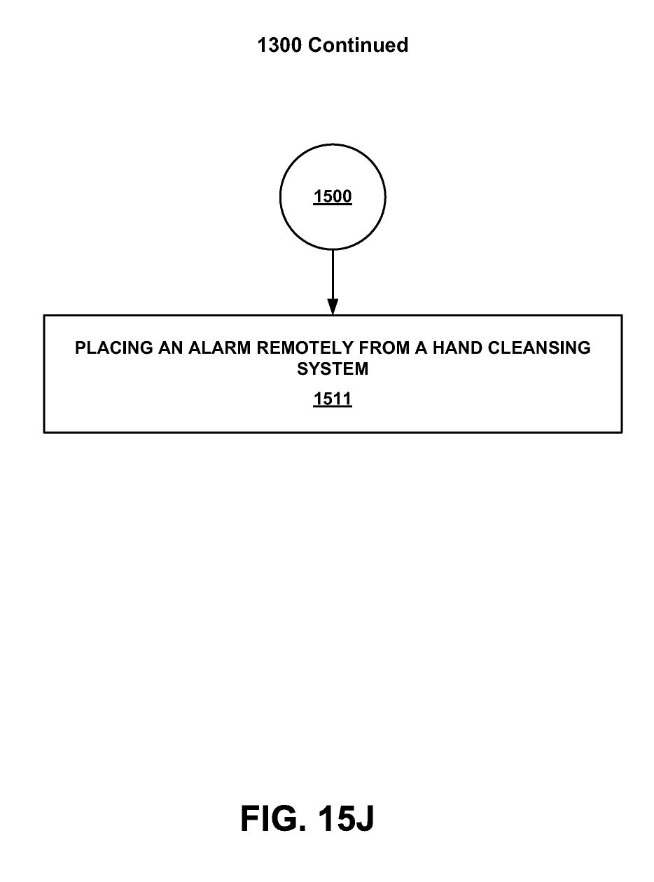

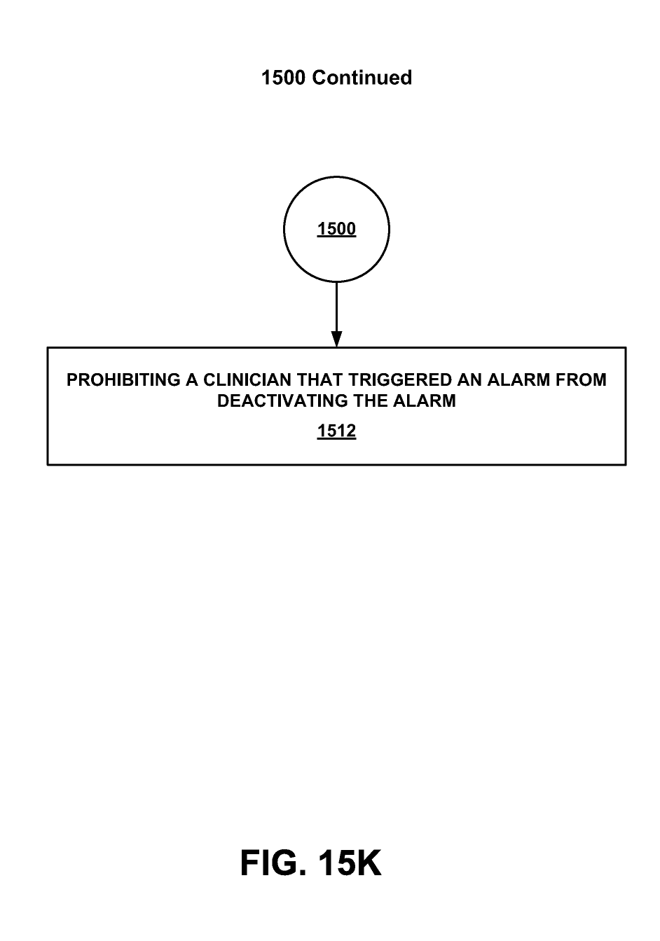

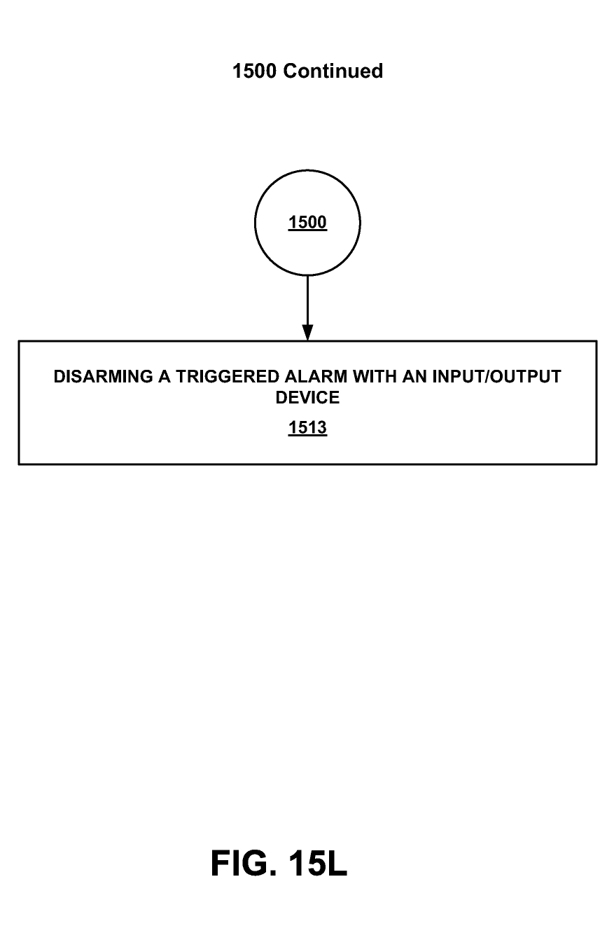

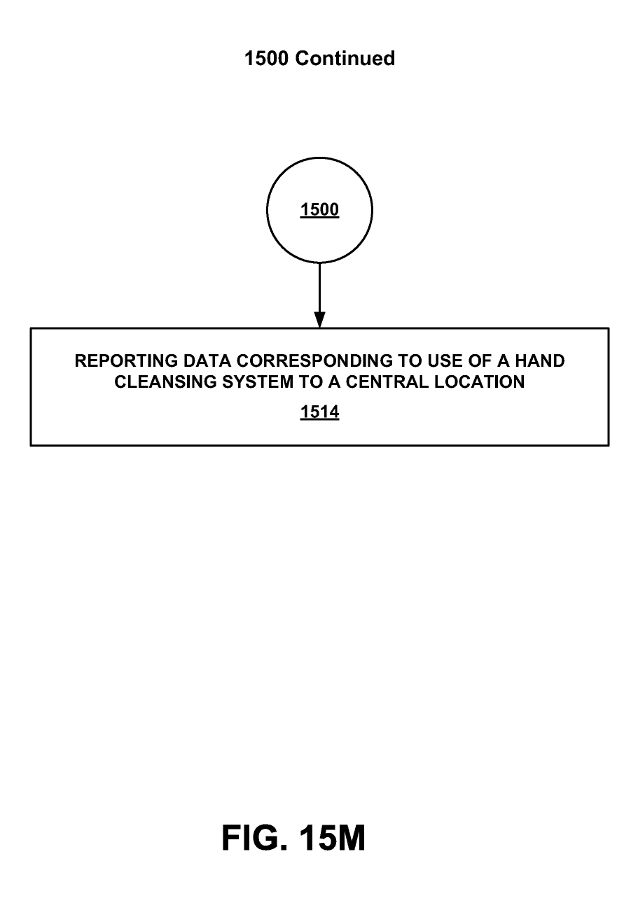

The following discussion sets forth in detail the operation of some example methods of operation of embodiments. With reference to FIGS. 15A-15M, flow diagram 1500 illustrates example procedures used by various embodiments. Flow diagram 1500 includes some procedures that, in various embodiments, are carried out by a processor under the control of computer-readable and computer-executable instructions. In this fashion, procedures described herein and in conjunction with flow diagram 1500 are or may be implemented using a computer, in various embodiments. The computer-readable and computer-executable instructions can reside in any tangible computer readable storage media, such as, for example, in data storage features such as RAM 608, ROM 610, and/or storage device 612 (all of FIG. 6). The computer-readable and computer-executable instructions, which reside on tangible computer readable storage media, are used to control or operate in conjunction with, for example, one or some combination of processor 606A, or other similar processor(s) 606B and 606C. Although specific procedures are disclosed in flow diagram 1500, such procedures are examples. That is, embodiments are well suited to performing various other procedures or variations of the procedures recited in flow diagram 1500. Likewise, in some embodiments, the procedures in flow diagram 1500 may be performed in an order different than presented and/or not all of the procedures described in this flow diagram may be performed. It is further appreciated that procedures described in flow diagram 1500 may be implemented in hardware, or a combination of hardware and firmware, or a combination of hardware.

With reference to FIGS. 15A-15M, flow diagram 1500 illustrates example procedures used by various embodiments. Flow diagram 1500 includes process and operations that, in various embodiments, are carried out by one or more of the electronic devices illustrated in at least FIG. 1, FIG. 3, or via a computer system 600.

Although specific procedures are disclosed in flow diagram 1500, such procedures are examples. That is, embodiments are well suited to performing various other operations or variations of the operations recited in the process of flow diagram 1500. Likewise, in some embodiments, the operations in flow diagram 1500 may be performed in an order different than presented, not all of the operations described in one or more of these flow drawings may be performed, and/or one or more additional operation may be added.

FIG. 15A is a flow diagram 1500 of an example method of detecting hand cleansing. Reference will be made to FIGS. 1-14 to facilitate the explanation of the operations of the method of flow diagram 1500. In one embodiment, the method of flow diagram 1500 describes determining the identity of clinician 700, determining whether clinician 700 needs to cleanse their hands 200, and whether clinician 700 cleansed their hands 200 following a particular protocol.

At operation 1501, in one embodiment, hand cleansing system 100 determines an identity of clinician 700 proximate hand cleansing system 100. As discussed above, in some embodiments a clinician 700 has a RFID tag or other identifying device which identification unit 120 uses to identify a clinician. In some embodiments, hand cleansing system 100 relays the identity of clinician 120 to hand cleansing data compiler 320 and/or central database 340. Central database 340, in some embodiments, can track how and when clinician 700 cleansed their hands, the amount of time clinician 700 cleansed their hands, whether clinician 700 cleansed their hands according a hand cleansing protocol, what hand cleansing agent 1220 was used, etc.

At operation 1502, in one embodiment, hand cleansing system 100 determines whether the clinician is washing at least one hand. Sensors 110 may be configured to detect motion, or may be configured to detect how many hands are being washed, the amount of time spent washing, whether hands are being washed according to a protocol, etc.

At operation 1503, in one embodiment, hand cleansing system 100 determines whether clinician 700 is following a hand cleansing protocol correctly. As discussed above, hand cleansing protocols may be programmed in the control unit of hand cleansing system 100 or at central database 340, for example.

At operation 1504, in one embodiment, hand cleansing system 100 alarms when clinician 700 does not follow a hand cleansing protocol. Alarm 160 may signal that a hand cleansing protocol was not followed by flashing a light, producing noise, etc. Alarm system 160 may be deactivated by a clinician at the hand cleansing system 100, or at another location. For example, when an alarm is triggered a clinician 700 may need to deactivate the alarm at a nurse station. In some embodiments, someone other than clinician 700 must deactivate the alarm either at the hand cleansing system or at a remote location.

At operation 1505, in one embodiment, hand cleansing system 100 outputs data corresponding to the use of hand cleansing system 100. In some embodiments, hand cleansing system may output data through telematic device 140 or I/O device 150. In some embodiments hand cleansing system 100 automatically transmits data to central system 340 through telematics device 140. In some embodiments, hand cleansing system may output data through a disk drive. In such cases, clinicians 700 or administrators may collect data from each hand cleansing system individually.

At operation 1506, in one embodiment, hand cleansing system 100 sends and/or receives data with telematics device 130. Telematics device 130 may transmit data wirelessly to a central database 340 or hand cleansing data compiler 320. Central database 340 and hand cleansing data compiler 320 may comprise another telematic device 310 configured to receive output data.

At operation 1507, in one embodiment, hand cleansing system 100 times the amount of time clinician 700 cleanses their hands. In some embodiments, hand cleansing system 100 may be configured to ensure a clinician 700 cleanses their hands for a first amount of time for a first type of patient, or for a second amount of time for a second type of patient. For example, hand cleansing system 100 may instruct a physician to cleanse their hands for 20 seconds when working with a patient with a broken arm, or for 90 seconds when working with a patient undergoing an invasive surgery.

At operation 1508, in one embodiment, hand cleansing system 100 dispenses a cleansing agent 1220. In some embodiments, hand cleansing system 100 can be configured to dispense a first type of hand cleansing agent for one type of patient, or a second type of hand cleansing agent for a second type of patient. Due to the increase in resistance to antibiotics, clinicians 700 may not want to use the same type of cleansing agent for every type of patient. For example, is a patient has a broken arm, hand cleanser dispenser 170 may dispense a soap. If a patient is undergoing an invasive surgery, hand cleanser dispenser 170 may dispense octenidine dihydrochloride or CHG.

At operation 1509, in one embodiment, hand cleansing system 100 detects when clinician 700 enters a room in which hand cleansing system 100 is located. Hand cleansing system may be coupled with room sensors 710 which detect when a clinician enters a room. Room sensors 710 may be configured to determine whether the person entering the room is a clinician 700 that needs to cleanse their hands. In some embodiments, hand cleansing system 100 may be configured to access central database 340 to determine whether clinician 700 recently cleansed their hands at another hand cleansing system 100. In some embodiments, after a clinician 700 enters a room, room sensor 710 may start a timer so that an alarm will sound if clinician 700 does not use hand cleansing system 100 within a particular amount of time.

At operation 1510, in one embodiment, hand cleansing system 100 determines whether clinician 700 is within a particular distance 720 from a hand cleansing system. For example, in some embodiments, hand cleansing system 100 determines whether clinician 700 is within a few feet from sink 190 while water is running.

At operation 1511, in one embodiment, alarm 160 is located remotely from hand cleansing system 100. In some embodiments alarm 160 may be located at a nurse station or other administrative location. This provides accountability by alerting others when a clinician 700 fails to follow hand cleansing protocols.

At operation 1512, in one embodiment, alarm 160 prohibits clinician 700 from deactivating alarm 160. In some embodiments, to ensure accountability, someone other than the clinician must disarm alarm 160. Another clinician 700, nurse, or administrator may need to go to hand cleansing system 100 and deactivate the alarm. In some embodiments, someone other than clinician 700 must deactivate alarm 160 at a nurses station, for example.

At operation 1513, in one embodiment, alarm 160 may be disarmed using I/O device 150. In some embodiments, alarm 160 may deactivated by an I/O device coupled with hand cleansing system 100 or on alarm 160 itself. In some embodiments, someone may deactivate alarm 160 by imputing a code into a keypad, signing a pad using a stylus, or through some other means of identification including biometrics such as voice, fingerprint, or iris.

At operation 1514, in one embodiment, hand cleansing system 100 reports data corresponding to the use of hand cleansing system 100 to a central database 340. As discussed above, central database 340 may compile and analyze all instances of hand cleansing. Central database 340 may be configured to contain information corresponding with: which clinicians 700 cleansed their hands 200, when clinicians 700 cleansed their hands 200, which hand cleansing system 100 a clinician used, whether clinicians 700 followed hand cleansing protocol, which cleansing agent 1220 was dispensed, how much of cleansing agent 1220 was dispensed, the average number of times a clinician 700 used a hand cleansing system 100, the average amount of time a clinician 700 spent cleansing their hands 200, how many rooms a clinician 700 visited, the amount of time between when a clinician entered a particular room and used the hand cleansing system 100 located in that room, the amount of time between when a clinician used a hand cleansing system 100 and entered a particular room, the number of alarms a particular clinician 700 triggered, the number of alarms all clinicians 700 triggered, the average amount of time all clinicians 700 spent using hand cleansing systems 100, etc.

Example embodiments of the subject matter are thus described. Although various embodiments of the subject matter have been described in a language specific to structural features and/or methodological acts, it is to be understood that the appended claims are not necessarily limited to the specific features or acts described above. Rather, the specific features and acts described above are disclosed as example forms of implementing the claims and their equivalents.

What is claimed is:

1. A hand cleansing system comprising:
at least one hand cleansing sensor, said hand cleansing sensor configured to detect whether at least one hand of a clinician is being cleansed; and
an identification unit coupled with said hand cleansing sensor configured to determine an identity of said clinician using said hand cleansing system when said hand cleansing system is in use.

2. The hand cleansing system of claim 1, further comprising:
an alarm.

3. The hand cleansing system of claim 1, further comprising:
an input/output device configured for receiving and delivering data from said clinician or an administrator.

4. The hand cleansing system of claim 1, further comprising:
a telematic device configured for sending and receiving data.

5. The hand cleansing system of claim 1, further comprising:
a timer configured for timing an amount of time said clinician cleanses their hands.

6. The hand cleansing system of claim 1, further comprising:
a cleansing agent dispenser configured for dispensing a cleansing agent.

7. The hand cleansing system of claim 1, further comprising:
a room sensor that detects when a clinician enters a room in which said hand cleansing is located.

8. The hand cleansing system of claim 1, further comprising:
a distance determinator configured for determining a distance between said clinician and said hand cleansing system.

9. The hand cleansing system of claim 1, further comprising:
a control unit configured for coordinating operations of said hand cleansing sensor, said identification unit, said alarm, said input/output device, said telematic device, said timer, said room sensor, and said distance determinator.

10. The hand cleansing system of claim 1, wherein said hand cleansing sensor can detect whether proper hand cleansing motions are performed.

11. The hand cleansing system of claim 1, wherein said alarm is located remotely from said hand cleansing system.

12. The hand cleansing system of claim 1, wherein said alarm may not be disarmed by a clinician that triggered said alarm.

13. The hand cleansing system of claim 1, wherein said input/output device is configured for disarming said alarm.

14. The hand cleansing system of claim 1, wherein said telematic device is configured for transmitting data relating to hand cleansing to a central location.

15. The hand cleansing system of claim 1, wherein said hand cleansing system is programmable.

16. A method for detecting hand cleansing comprising:
determining an identity of a clinician proximate a hand cleansing system; and
determining whether said clinician is cleansing at least one hand.

17. The method of claim 16, further comprising:
determining whether said clinician is following a hand cleansing protocol correctly.

18. The method of claim 16, further comprising:
alarming when said clinician does not follow said hand cleansing protocol.

19. The method of claim 16, further comprising:
outputting data corresponding to use of said hand cleansing system.

20. The method of claim 16, further comprising:
sending and receiving data with a telematic device.

21. The method of claim 16, further comprising:
timing an amount of time said clinician cleanses their hands.

22. The method of claim 16, further comprising:
dispensing a cleansing agent.

23. The method of claim 16, further comprising:
detecting when said clinician enters a room in which said hand cleansing system is located.

24. The method of claim 16, further comprising:
determining whether said clinician is within a particular distance from said hand cleansing system.

25. The method of claim 16, further comprising:
placing said alarm remotely from said hand cleansing system.

26. The method of claim 16, further comprising:
prohibiting said clinician that triggered said alarm from deactivating said alarm.

27. The method of claim 16, further comprising:
disarming a triggered alarm with an input/output device.

28. The method of claim 16, further comprising:
reporting data corresponding to use of said hand cleansing system to a central location.

29. A method for ensuring adherence to a hand cleansing protocol comprising:
determining the identity of a clinician proximate a hand cleansing system;
determining whether said clinician cleanses at least one hand according to said protocol; and
outputting data corresponding to use of said hand cleansing system.

30. The method of claim 29, further comprising:
reporting said data to a central location.

31. A hand cleansing apparatus comprising:
at least one hand cleansing sensor, configured to detect whether a clinician is utilizing said hand cleansing apparatus;
an identification unit coupled with said hand cleansing sensor, said identification unit configured to determine the identity of said clinician proximate said hand cleansing system; and
an alarm coupled to said hand cleansing sensor, said alarm configured to provide an indicator that a hand cleansing protocol was not followed correctly.

32. The hand cleansing apparatus of claim 31, further comprising:
an input/output device configured for receiving and delivering data from said clinician or an administrator.

33. The hand cleansing apparatus of claim 31, further comprising:
a telematic device configured for sending and receiving data.

34. The hand cleansing apparatus of claim 31, further comprising:
a timer configured for timing an amount of time said clinician cleanses their hands.

35. The hand cleansing apparatus of claim 31, further comprising:
a cleansing agent dispenser configured for dispensing a cleansing agent.

36. The hand cleansing apparatus of claim 31, further comprising:
a room sensor that detects when said clinician enters a room in which said hand cleansing apparatus is located.

37. The hand cleansing system of claim 1, further comprising:
a distance determinator configured for determining a distance between said clinician and said hand cleansing system.

38. The hand cleansing apparatus of claim 31, further comprising:
a control unit configured for coordinating operations of said hand cleansing sensor, said identification unit, said alarm, said input/output device, said telematic device, said timer, said cleansing agent dispenser, and said room sensor.

39. The hand cleansing apparatus of claim 31, wherein said hand cleansing sensor can detect whether proper hand cleansing motions are performed.

40. The hand cleansing apparatus of claim 31, wherein a distance determinator determines a distance between said clinician and said hand cleansing system.

41. The hand cleansing apparatus of claim 31, wherein said alarm is located remotely from said hand cleansing system.

42. The hand cleansing apparatus of claim 31, wherein said alarm may not be disarmed by a clinician that triggered said alarm.

43. The hand cleansing apparatus of claim 31, wherein said input/output device is configured for disarming a triggered alarm.

44. The hand cleansing apparatus of claim 31, wherein said telematic device is configured for transmitting data relating to hand cleansing data to a hospital system, wherein said hospital system maintains and analyzes said data.

45. The hand cleansing apparatus of claim 31, wherein said hand cleansing apparatus outputs data corresponding with use of said hand cleansing apparatus including:
amount of time spent cleansing hands, type of said cleansing agent(s) used, amount of said cleansing agent(s) used, whether said alarm was triggered, identity of said clinician or said administer that deactivated said alarm, amount of time spent in room where said hand cleansing system is located before cleansing hands, amount of time since previous hand cleansing, and amount of time before subsequent hand cleansing.

46. The hand cleansing apparatus of claim 31, wherein:
said hand cleansing apparatus is programmable.

47. A hand cleansing apparatus comprising:
a plurality of dispensers; and
a plurality of cleansing agents, each of said plurality of cleansing agents dispensed within each of said plurality of dispensers.

48. The hand cleansing apparatus of claim 48 wherein said cleansing agent comprises a cleansing fluid.

49. The hand cleansing apparatus of claim 48 wherein said cleansing agent comprises an alcohol based antiseptic.

50. The hand cleansing apparatus of claim 49 wherein said cleansing agent prevents the formation of biofilms when said cleansing agent is dispensed.

51. The hand cleansing apparatus of claim 49 wherein said antiseptic is chlorhexidine gluconate.

52. The hand cleansing apparatus of claim 49 wherein said antiseptic is also antimicrobial.

53. The hand cleansing apparatus of claim 52 wherein said antiseptic is also anti-irritative.

54. The hand cleansing apparatus of claim 49 wherein said antiseptic is octenidine dihydrochloride.

55. The hand cleansing apparatus of claim 48 wherein said cleansing agent is water.
